(19) United States
(12) Patent Application Publication
(10) Pub. No.: US 2021/0206662 A1
(43) Pub. Date: Jul. 8, 2021

(54) WATER STERILIZING TUBE

(71) Applicant: High Power Lighting Corp., New Taipei City (TW)

(72) Inventors: Chih-Hung WEI, New Taipei City (TW); Ming-Chang WU, New Taipei City (TW); Jian-Yang WU, New Taipei City (TW); Guan-Yao HUANG, New Taipei City (TW); Po-Ning CHANG, New Taipei City (TW); Wun-Ru LIAO, New Taipei City (TW); Chih-Yang HSU, New Taipei City (TW)

(21) Appl. No.: 16/819,584

(22) Filed: Mar. 16, 2020

(30) Foreign Application Priority Data
Jan. 3, 2020 (TW) 109100243

(51) Int. Cl. C02F 1/32

(52) U.S. Cl. CPC C02F 1/325; C02F 2201/3222; C02F 2303/04; C02F 2201/328; C02F 2201/003; C02F 2201/3228

(57) ABSTRACT

A water sterilizing tube includes a shell, an outer tube, an inner tube and a UV lighting module. The shell has a first end, a second end and an annular wall. The first end is provided with a water inlet and a water outlet. The outer tube is coaxially disposed in the shell. An end of the outer tube communicates with the water outlet. An air gap is formed between the outer tube and the annular wall. The inner tube is coaxially disposed in the outer tube. An end of the inner tube communicates with the water inlet. An extended channel is formed between the outer and inner tubes. The UV lighting module has a UV light corresponding to the outer tube. An axis line of the outer and the inner tube and a direction of light emitting of the UV light are superposed with each other.

### BACKGROUND OF THE INVENTION

### Technical Field

The invention relates to water purifiers, particularly to water sterilizing tubes.

### Related Art

Tap water must contain chlorine to keep the microbial index safe. However, chlorine reacting with organic acids will produce carcinogens such as chloroform. Thus, chlorine in drinking water must be filtered out. However, water pipelines without chlorine easily breed microorganisms such as bacteria, fungi and mold to make filtered drinking water unsafe.

Ultraviolet (UV) rays can destroy nucleic acid, DNA, RNA of microorganisms to make disinfection without toxic by-products. As a result, how to apply UV rays into water pipelines and increase utilization efficiency of UV rays are important issues for the industry.

### SUMMARY OF THE INVENTION

An object of the invention is to provide a water sterilizing tube, which can improve disinfectant ability to water by both an extended channel between a light-permeable quartz inner tube and a light-permeable quartz outer tube and an air gap between the light-permeable quartz outer tube and an annular wall.

To accomplish the above object, the invention provides a water sterilizing tube which includes: a shell, having a first end, a second end opposite to the first end and an annular wall between the first and second ends, and the first end being provided with a water inlet and a water outlet; a light-permeable quartz outer tube, coaxially disposed in the shell, an end of the light-permeable quartz outer tube communicating with the water outlet and another end thereof being formed into a closed portion corresponding to the second end, and an air gap being formed between the light-permeable quartz outer tube and the annular wall; a light-permeable quartz inner tube, coaxially disposed in the light-permeable quartz outer tube, an end of the light-permeable quartz inner tube communicating with the water inlet and another end thereof being formed into an opening corresponding to the closed portion, and an extended channel being formed between the light-permeable quartz outer tube and the light-permeable quartz inner tube; and an ultraviolet (UV) lighting module, disposed between the second end and the closed portion, comprising a UV light corresponding to the light-permeable quartz outer tube, an axis line of the light-permeable quartz outer tube, an axis line of the light-permeable quartz inner tube and a direction of light emitting of the UV light being superposed with each other.

By the air gap between the light-permeable quartz outer tube and the annular wall, the inside of the light-permeable quartz outer tube forms a high UV illumination status to effectively increase disinfectant efficiency to water.

By the extended channel between the light-permeable quartz inner tube and the light-permeable quartz outer tube, the continuance time of the water in the pipeline can be extended to enhance the radiation efficiency.

### BRIEF DESCRIPTION OF THE DRAWINGS

FIG. 1 is an exploded view of the water sterilizing tube of the invention;

FIG. 2 is an assembled view of the water sterilizing tube of the invention;

FIG. 3 is a schematic cross-section view of the water sterilizing tube of the invention;

FIG. 4 is a schematic view of the water sterilizing tube of the invention in use; and

FIG. 5 is a schematic cross-section view of another embodiment of the water sterilizing tube of the invention.

### DETAILED DESCRIPTION OF THE INVENTION

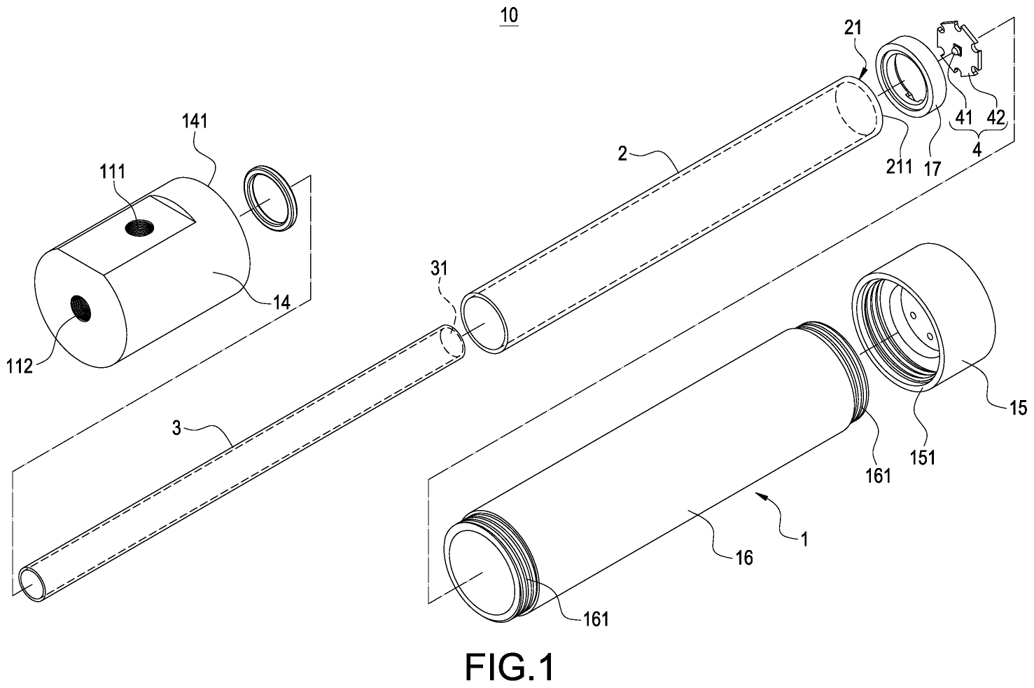

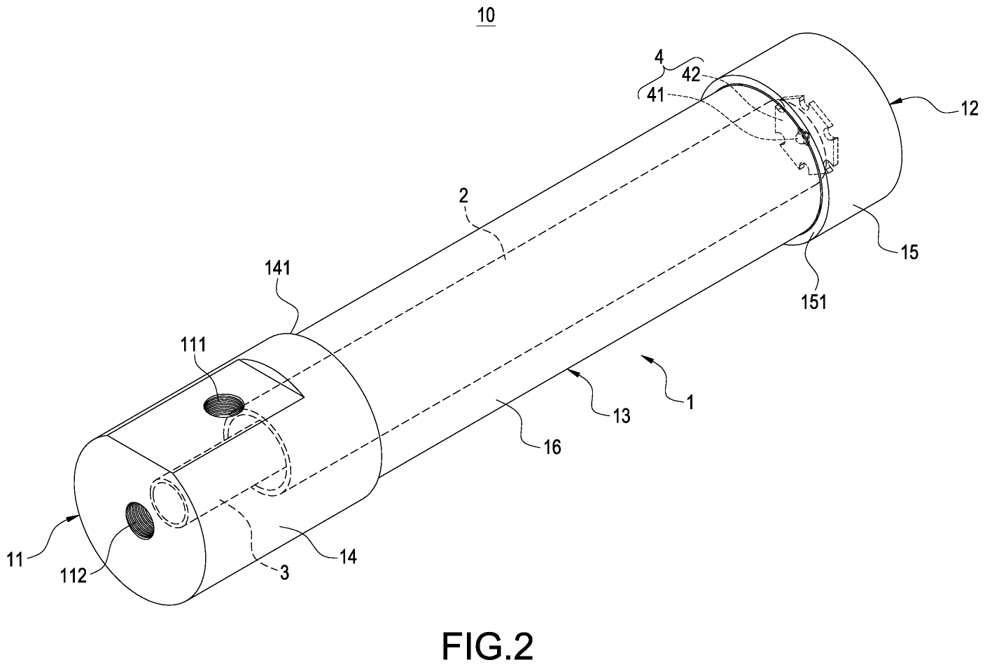

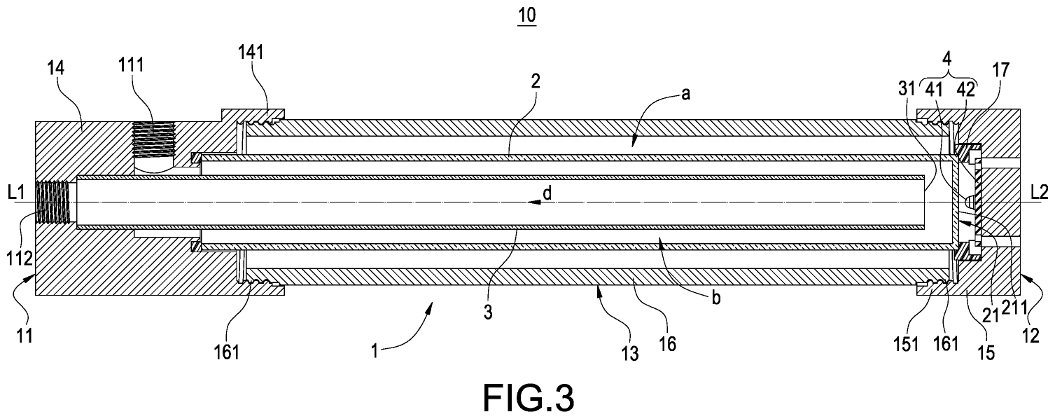

Please refer to FIGS. 1-4. The invention provides a water sterilizing tube 10 including a shell 1, a light-permeable quartz outer tube 2, a light-permeable quartz inner tube 3 and an ultraviolet (UV) lighting module 4.

The shell 1 has a first end 11, a second end 12 opposite to the first end 11 and an annular wall 13 between the first end 11 and the second end 12. The first end 11 is provided with a water inlet 112 and a water outlet 111. The shell 1 may be made of an opaque material, but not limited to this.

In detail, the shell 1 in this embodiment includes a first fixer 14, a second fixer 15 and a tube 16 connected between the first fixer 14 and the second fixer 15. The water outlet 111 and the water inlet 112 are located in the first fixer 14 and the annular wall 13 is formed on the tube 16.

In addition, each of the first and second fixers 14, 15 is extended with a threaded ring 141, 151. Each of two ends of the tube 16 is provided with a thread section 161. The thread sections 161 are separately screwed with the threaded rings 141, 151 to make the tube 16 firmly connected between the first fixer 14 and the second fixer 15. The tube 16, the first fixer 14 and the second fixer 15 are made of metal, Teflon and plastic, respectively, but not limited to these.

As shown in FIGS. 1-4, the light-permeable quartz outer tube 2 is coaxially disposed in the shell 1. An end of the light-permeable quartz outer tube 2 is connected to the first fixer 14 and communicates with the water outlet 111. The other end thereof is formed into a closed portion 21 corresponding to the second end 12. An air gap “a” is formed between the light-permeable quartz outer tube 2 and the annular wall 13. The air gap “a” is formed by air between the light-permeable quartz outer tube 2 and the annular wall 13. A water-proof gasket is sandwiched between the light-permeable quartz outer tube 2 and the first fixer 14. In this embodiment, the closed portion 21 is formed by a closed end 211 of the light-permeable quartz outer tube 2, which is adjacent to the second end 12.

As shown in FIGS. 1-4, the light-permeable quartz inner tube 3 is coaxially disposed in the light-permeable quartz outer tube 2. An end of the light-permeable quartz inner tube 3 is connected to the first fixer 14 and communicates with the water inlet 112. The other end thereof is formed into an opening 31 corresponding to the closed portion 21. An extended channel “b” is formed between the light-permeable quartz outer tube 2 and the light-permeable quartz inner tube 3. As a result, drinking water flows into the light-permeable quartz inner tube 3 from the water inlet 112. Because the closed portion 21 of the light-permeable quartz outer tube 2 can block the drinking water to guide the drinking water to flow to the extended channel “b”, finally the drinking water flows out of the water outlet 111 through the extended channel “b”.

As shown in FIGS. 1-4, the ultraviolet (UV) lighting module 4 is disposed between the second end 12 and the closed portion 21 and includes a UV light 41 and a circuit board 42. The UV light 41 irradiates the light-permeable quartz outer tube 2 to accomplish a disinfection function. The circuit board 42 is fixed on the second fixer 15 and the UV light 41 is fixed to and electrically connected to the circuit board 42. The UV light 41 is a low-pressure mercury lamp or a light-emitting diode (LED). Ultraviolet (UV) rays from the UV light 41 can destroy nucleic acid, DNA, RNA of microorganisms to make disinfection without toxic by-products.

In addition, an axis line L1 of the light-permeable quartz outer tube 2, an axis line L2 of the light-permeable quartz inner tube 3 and a light emitting direction d of the UV light 41 are superposed with each other. In other words, the light-permeable quartz outer tube 2 and the light-permeable quartz inner tube 3 are coaxially arranged, and the UV light 41 irradiate the axis lines of the light-permeable quartz outer tube 2 and the light-permeable quartz inner tube 3.

Further, the shell 1 includes a connecting member 17 sandwiched between the second fixer 15 and the light-permeable quartz outer tube 2 and surrounding the UV lighting module 4. The closed end 211 is inserted and positioned into the connecting member 17. The connecting member 17 is made of a UV-resistant material such as Teflon, metal or plastic with a UV-resistant surface. The first fixer 14 is also irradiated by UV light, so the first fixer 14 is preferably made of a UV-resistant material such as Teflon, metal or plastic with a UV-resistant surface. The circuit board 42 is mounted in the second fixer 15, so the second fixer 15 is preferably made of heat-dissipating metal.

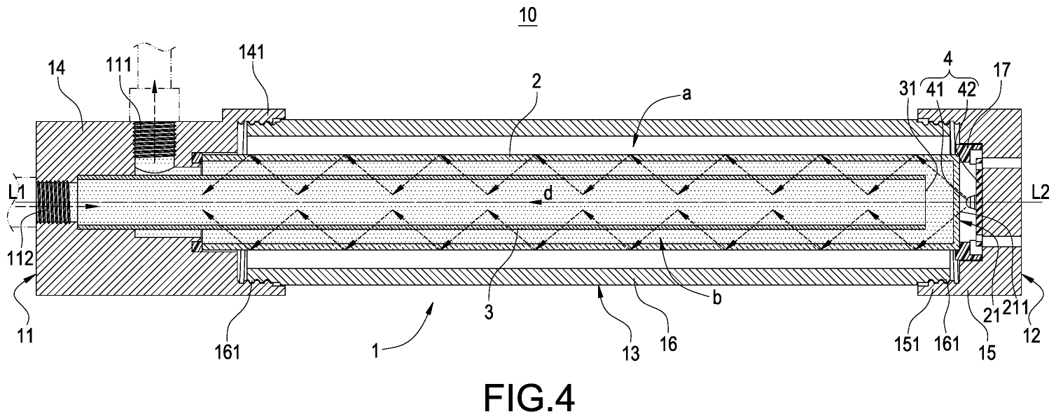

As shown in FIG. 4, which is a schematic view of the water sterilizing tube 10 of the invention in use, drinking water flows from the water inlet 112 into the light-permeable quartz inner tube 3, and flows out of the water outlet 111 via the opening 31 and the extended channel “b”. Also, an axis line L1 of the light-permeable quartz outer tube 2, an axis line L2 of the light-permeable quartz inner tube 3 and a light emitting direction d of the UV light 41 are superposed with each other, so most of the UV light from the UV light 41 can be refracted by the closed portion 21 to enter the light-permeable quartz outer tube 2 to irradiate the drinking water. Thus, the drinking water can be disinfected.

Additionally, the air gap “a” is formed between the light-permeable quartz outer tube 2 and the annular wall 13. Because the refractive index of the light-permeable quartz tube is greater than the refractive index of air, a refractive angle is greater than an incident angle when light travels from a material with a high refractive index to a material with a low refractive index. As a result, the light cannot enter the material with a low refractive index to generate total reflection and a better reflective effect can be obtained. UV light can be totally reflected in the light-permeable quartz outer tube 2 almost without loss so as to make the inside of the light-permeable quartz outer tube 2 be in a status of high UV illumination. Thus, the disinfection efficiency of the water sterilizing tube 10 can be increased.

By the extended channel “b” between the light-permeable quartz inner tube 3 and the light-permeable quartz outer tube 2, the continuance time of the water in the pipeline can be extended to enhance the radiation efficiency of the water sterilizing tube 10.

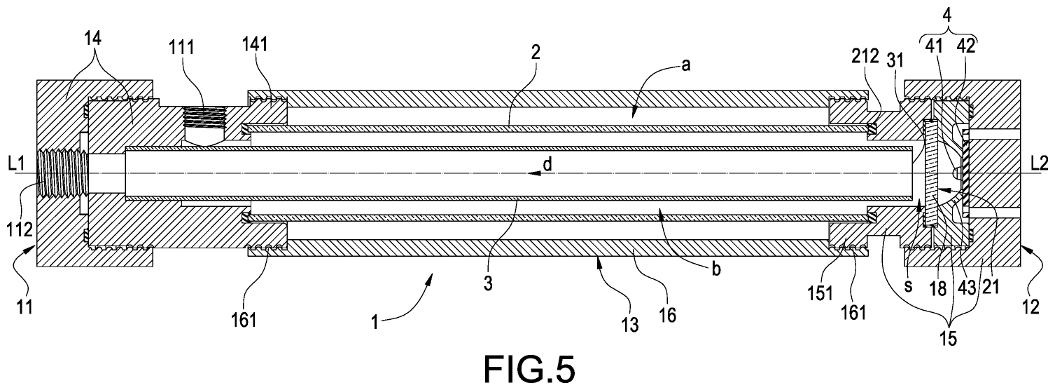

Please refer to FIG. 5, which shows another embodiment of the water sterilizing tube 10 of the invention. The embodiment shown in FIG. 5 is substantially identical to the embodiment shown in FIGS. 1-4. The embodiment shown in FIG. 5 differs from the embodiment shown in FIGS. 1-4 by an open end 212 formed at an end of the light-permeable quartz outer tube 2, which is adjacent to the second end 12. The closed portion 21 is constituted by a closed space s encompassed by the open end 212 and the second end 12.

In detail, the open end 212 is inserted into the second fixer 15. A water-proof gasket is sandwiched between the open end 212 and the second fixer 15. The second fixer 15 is provided with a quartz closer 18 sandwiched between the opening 31 and the UV light 41. In this embodiment, the closed portion 21 is formed by a closed end 211 of the light-permeable quartz outer tube 2, which is adjacent to the second end 12. The closed space s is formed between the second fixer 15, the quartz closer 18, the opening 31 and the open end 212. Thereby, most of the UV light from the UV light 41 can be refracted by the quartz closer 18 to enter the light-permeable quartz outer tube 2 to irradiate the drinking water. Thus, the drinking water can be disinfected. Also, when drinking water flows into the light-permeable quartz inner tube 3 from the water inlet 112, both the second fixer 15 and the quartz closer 18 can block the drinking water to guide the drinking water to flow to the extended channel “b” via the opening 31, finally the drinking water flows out of the water outlet 111 through the extended channel “b”.

In addition, the UV lighting module 4 further includes a reflecting cup 43 surrounding the UV light 41 and sandwiched between the closed portion 21 and the circuit board 42 so as to make most of UV light stably reflected into the light-permeable quartz inner tube 3.

In this embodiment, the first fixer 14 is composed of pipes to be convenient to manufacture the water outlet 111 and the water inlet 112. The second fixer 15 is composed of pipes to be convenient to clamp the quartz closer 18, but not limited to this.

The reflecting cup 43 is made of a UV-resistant material such as Teflon, metal or plastic with a UV-resistant surface. The first fixer 14 is also irradiated by UV light, so the first fixer 14 is preferably made of a UV-resistant material such as Teflon, metal or plastic with a UV-resistant surface. The circuit board 42 is mounted in the second fixer 15, so the second fixer 15 is preferably made of heat-dissipating metal. The portion of the second fixer 15, which is irradiated by UV light, is made of a UV-resistant material such as Teflon, metal or plastic with a UV-resistant surface.

It will be appreciated by persons skilled in the art that the above embodiments have been described by way of example only and not in any limitative sense, and that various alterations and modifications are possible without departure from the scope of the invention as defined by the appended claims.

What is claimed is:

1. A water sterilizing tube comprising:
a shell, having a first end, a second end opposite to the first end and an annular wall between the first and second ends, and the first end being provided with a water inlet and a water outlet;
a light-permeable quartz outer tube, coaxially disposed in the shell, an end of the light-permeable quartz outer tube communicating with the water outlet and another end thereof being formed into a closed portion corresponding to the second end, and an air gap being formed between the light-permeable quartz outer tube and the annular wall;
a light-permeable quartz inner tube, coaxially disposed in the light-permeable quartz outer tube, an end of the light-permeable quartz inner tube communicating with the water inlet and another end thereof being formed into an opening corresponding to the closed portion, and an extended channel being formed between the light-permeable quartz outer tube and the light-permeable quartz inner tube; and
an ultraviolet (UV) lighting module, disposed between the second end and the closed portion, comprising a UV light corresponding to the light-permeable quartz outer tube, an axis line of the light-permeable quartz outer tube, an axis line of the light-permeable quartz inner tube and a direction of light emitting of the UV light being superposed with each other.

2. The water sterilizing tube of claim 1, wherein the shell comprises a first fixer, a second fixer and a tube connected between the first fixer and the second fixer, the water outlet and the water inlet are located in the first fixer, and the annular wall is formed on the tube.

3. The water sterilizing tube of claim 2, wherein both an end of the light-permeable quartz outer tube and an end of the light-permeable quartz inner tube are inserted into the first fixer.

4. The water sterilizing tube of claim 2, wherein each of the first and second fixers is extended with a threaded ring, each of two ends of the tube is provided with a thread section, and the thread sections are separately screwed with the threaded rings.

5. The water sterilizing tube of claim 2, wherein the UV lighting module comprises a circuit board electrically connected to the UV light.

6. The water sterilizing tube of claim 5, wherein the closed portion is formed by a closed end of the light-permeable quartz outer tube, which is adjacent to the second end.

7. The water sterilizing tube of claim 6, wherein the shell further comprises a connecting member sandwiched between the second fixer and the light-permeable quartz outer tube and surrounding the UV lighting module, the closed end is inserted into the connecting member, and the connecting member is made of a UV-resistant material.

8. The water sterilizing tube of claim 5, wherein an open end is formed at an end of the light-permeable quartz outer tube, which is adjacent to the second end, and the closed portion is constituted by a closed space encompassed by the open end and the second end.

9. The water sterilizing tube of claim 8, wherein the open end is inserted into the second fixer, the second fixer is provided with a quartz closer sandwiched between the opening and the UV light, and the closed space is formed between the second fixer, the quartz closer, the opening and the open end.

10. The water sterilizing tube of claim 5, wherein the UV lighting module further comprises a reflecting cup surrounding the UV light and sandwiched between the closed portion and the circuit board.
